(19) United States
(12) Patent Application Publication
(10) Pub. No.: US 2024/0225958 A1
(43) Pub. Date: Jul. 11, 2024

(54) KIT AND METHOD FOR ADMINISTERING AN ACTIVE INGREDIENT TO ELICIT A PHYSIOLOGICAL RESPONSE

(71) Applicant: ALLERGY UNLIMITED, LLC, Findlay, OH (US)

(72) Inventor: Amber Patterson, Findlay, OH (US)

(21) Appl. No.: 18/408,158

(22) Filed: Jan. 9, 2024

Related U.S. Application Data
(60) Provisional application No. 63/479,202, filed on Jan. 10, 2023.

(51) Int. Cl. A61J 1/20; A61J 1/16; A61K 39/00; A61K 39/36; A61K 39/39

(52) U.S. Cl. CPC A61J 1/2089; A61J 1/165; A61K 39/36; A61K 39/39; A61K 2039/54; A61K 2039/55505; A61K 2039/577

(57) ABSTRACT

A kit and method are provided for administering an active ingredient to a subject to elicit a response. The kit comprises a first container with an active ingredient and a second container with an adjuvant. The containers permit the ingredients to be admixed to form a compounded preparation for delivery into the subject's tissue. The active ingredient can comprise an allergen. The kit can also include temperature control and mixing components. The method comprises providing the kit, admixing the ingredients, and injecting the compounded form into the subject's tissue such as a lymph node. The injection can be monitored and repeated. The approach can elicit various responses such as desensitization according to the type of active ingredient used. Overall, the kit and method provide targeted delivery of active ingredients to efficiently provoke a localized response in the subject.

### CROSS-REFERENCE TO RELATED APPLICATIONS

This application claims the benefit of U.S. Provisional Application No. 63/479,202, filed on Jan. 10, 2023. The entire disclosure of the above application is incorporated herein by reference.

### FIELD

The present technology relates to articles of manufacture, systems, kits, and processes for eliciting a physiological response in a subject upon administering an active ingredient.

### INTRODUCTION

This section provides background information related to the present disclosure which is not necessarily prior art.

Various approaches have been developed for administering active ingredients to various subjects, such as a human, in order to elicit a desired response. These approaches typically involve the use of pharmaceutical compositions or formulations that combine the active ingredient with other substances, such as excipients or carriers, to facilitate delivery and enhance the therapeutic effect. However, these conventional approaches often suffer from limitations in terms of the flexibility and convenience of administration, as well as the ability to achieve targeted delivery directly into the tissue of the subject.

Ways to administer active ingredients often take one of three common approaches. One way involves the use of pre-formulated pharmaceutical compositions, where the active ingredient and adjuvant are already combined in a single container. These compositions are typically prepared in advance and stored until needed. While this approach offers convenience, it cannot provide the desired flexibility in terms of adjusting the dosage or the ratio of active ingredient to adjuvant. Additionally, the pre-formulated compositions cannot allow for immediate admixing of the active ingredient and adjuvant prior to administration, which can limit the ability to achieve optimal therapeutic outcomes. Another way involves the use of separate containers for the active ingredient and adjuvant, allowing for on-demand admixing prior to administration. However, these approaches often require additional steps and equipment for compounding the active ingredient and adjuvant, which can be time-consuming and cumbersome. Furthermore, the ability to deliver the compounded form directly into the tissue of the subject in a controlled and efficient manner can be limited. A third way involves immunotherapy to provide a treatment for a variety of conditions including allergies. An allergy is the result of an immune system reaction against a substance that should normally be inoffensive to the host.

The treatment duration for conventional immunotherapy can unfortunately be long and time consuming and can comprise dozens if not hundreds of allergen injections or shots, each requiring an hour or more of strict medical supervision after the shot is administered. For desensitization to certain allergens which are known to cause severe side effects, such as insect venom cat hair or dust mites, patients must remain in the doctor's office for an hour after each injection for observation. Thus, the medical and economic costs are very high for this type of treatment.

Another technique for modulation of an immune response is described in U.S. Pat. No. 6,773,695 to Thomas M. Kündig. The Kündig patent discloses the modulation or elimination of an allergic condition can be achieved by injecting small amounts of allergen directly into a lymph node, which greatly reduces the potential for side effects. This technique is known as intralymphatic immunotherapy. The injection procedure is performed in a comfortable outpatient setting under ultrasound guidance. Research has shown intralymphatic immunotherapy injections to be similar in effectiveness to conventional immunotherapy and virtually painless. Most patients begin noticing improvement in their symptoms within a few months of completing the treatment experience. Advantages compared to conventional allergy shots include short treatment duration and enhanced safety.

There is a need for a kit and method for safely and effectively providing an active ingredient for eliciting a physiological response in a subject, such as a human subject.

### SUMMARY

In concordance with the instant disclosure, a kit and method for safely and effectively providing an active ingredient for eliciting a physiological response in a subject, has surprisingly been discovered. The present technology includes articles of manufacture, systems, kits, and processes for eliciting a response in a subject upon administering an active ingredient.

In certain embodiments, the present disclosure provides a kit designed for the administration of an active ingredient to a subject with the purpose of eliciting a specific response. This kit comprises two distinct containers: the first container is filled with the active ingredient itself, while the second container holds an adjuvant. These containers are specially designed to allow the contents to be mixed together, forming a compounded form. Once mixed, this compounded form is ready for direct delivery into the subject's tissue, which is done in such a way as to effectively elicit the desired response.

In certain embodiments, the present disclosure encompasses a method for administering an active ingredient to a subject in order to elicit a particular response. This method involves the use of a kit that includes two separate containers: the first container contains the active ingredient, and the second contains an adjuvant. The method includes the steps of mixing the active ingredient with the adjuvant to create a compounded form, and then administering this form directly into the subject's tissue by injection. This method is designed to ensure that the active ingredient is delivered in a manner that is sufficient to elicit the intended response from the subject.

In certain embodiments, the present disclosure presents a comprehensive kit for the targeted delivery of an active ingredient to a subject, aiming to elicit a specific physiological response. This kit includes a first container, which houses the active ingredient that can comprise allergens such as pollen, animal dander, venom, dust mites, or other substances. Accompanying this, a second container contains an adjuvant, potentially a physiologically acceptable carrier like alum, to enhance the response. Both containers are engineered to allow the active ingredient and the adjuvant to be combined, creating a compounded form. The first container is also designed with temperature control capabilities, ensuring that the active ingredient can be stored at a predetermined temperature, ranging from about 2° C. to about 8° C., for a minimum of 12 hours, and up to 24 hours or more. Additionally, the kit can include a third container that provides temperature control with a cooling medium such as water ice, dry ice, or a pre-refrigerated cooling gel pack. The proportions of the active ingredient and the adjuvant are meticulously configured so that the adjuvant constitutes about 5% to 50% of the total compounded form upon mixing. For the administration of this compounded form, the kit further comprises a syringe equipped with an injection needle, or alternatively, a dual-chambered syringe that simplifies the mixing process. This syringe facilitates the direct injection of the compounded form into the subject's tissue, which can include a lymph node, to efficiently elicit the desired response.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations and are not intended to limit the scope of the present disclosure.

FIG. 1 is a block diagram illustrating a kit, according to some embodiments of the present disclosure.

FIG. 2 is a flowchart illustrating a method for administering an active ingredient to a subject to elicit a response, according to some embodiments of the present disclosure.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as can be filed claiming priority to this application, or patents issuing therefrom. Regarding methods disclosed, the order of the steps presented is exemplary in nature, and thus, the order of the steps can be different in various embodiments, including where certain steps can be simultaneously performed, unless expressly stated otherwise. “A” and “an” as used herein indicate “at least one” of the item is present; a plurality of such items can be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that can arise from ordinary methods of measuring or using such parameters.

All documents, including patents, patent applications, and scientific literature cited in this detailed description are incorporated herein by reference, unless otherwise expressly indicated. Where any conflict or ambiguity can exist between a document incorporated by reference and this detailed description, the present detailed description controls.

Although the open-ended term “comprising,” as a synonym of non-restrictive terms such as including, containing, or having, is used herein to describe and claim embodiments of the present technology, embodiments can alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that can be recited in the art, even though element D is not explicitly described as being excluded herein.

As referred to herein, all compositional percentages are by weight of the total composition, unless otherwise specified. Disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter can define endpoints for a range of values that can be claimed for the parameter. For example, if Parameter X is exemplified herein to have value A and also exemplified to have value Z, it is envisioned that Parameter X can have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X can have other ranges of values including 1-9, 1-8, 1-3, 1-2, 2-10, 2-8, 2-3, 3-10, 3-9, and so on.

When an element or layer is referred to as being “on,” “engaged to,” “connected to,” or “coupled to” another element or layer, it can be directly on, engaged, connected or coupled to the other element or layer, or intervening elements or layers can be present. In contrast, when an element is referred to as being “directly on,” “directly engaged to,” “directly connected to” or “directly coupled to” another element or layer, there can be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. can be used herein to describe various elements, components, regions, layers and/or sections, these elements, components, regions, layers and/or sections should not be limited by these terms. These terms can be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, can be used herein for ease of description to describe one element or feature's relationship to another element(s) or feature(s) as illustrated in the figures. Spatially relative terms can be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device can be otherwise oriented (rotated 90 degrees or at other orientations) and the spatially relative descriptors used herein interpreted accordingly.

The present technology improves upon known intralymphatic immunotherapy techniques, including the technique for modulation of immune response as described in U.S. Pat. No. 6,773,695 to Thomas M. Kündig, the entire disclosure of which is incorporated herein by reference.

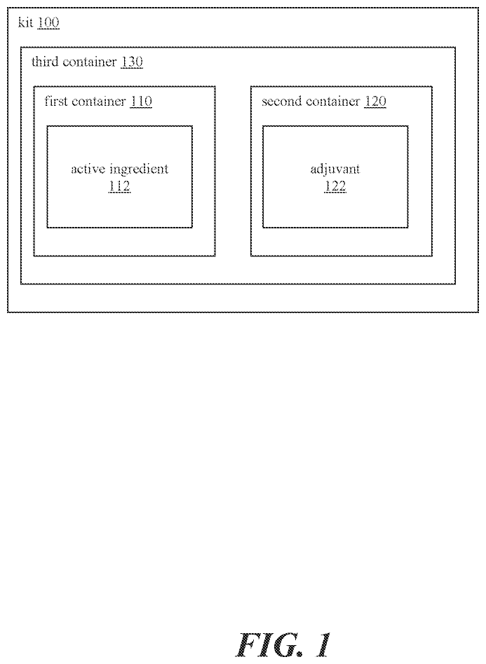

FIG. 1 is a block diagram that describes a kit 100, according to some embodiments of the present disclosure. The kit 100 can include a first container 110 and a second container 120. The first container 110 can include an active ingredient 112. The second container 120 can include an adjuvant 122. The first container 110 and the second container permit for the active ingredient 112 and the adjuvant 122 to be admixed to prepare a compounded form. In one non-limiting example, the compounded form can be suitable for delivery of the active ingredient directly into a tissue of the subject in a manner sufficient to elicit a desired physiological response from the subject.

As non-limiting examples, the active ingredient 112 can be an allergen, a hormone, or a hormone analog. Other suitable types of the active ingredients 112 can also be selected and used within the scope of the present disclosure and are discussed in greater detail herein. As used herein, an adjuvant is a drug or other substance, or a combination of substances, that is used to increase the efficacy or potency of certain active ingredients. The adjuvant 122 can be a physiologically acceptable carrier. The adjuvant 122 can be alum. It should be understood that the adjuvant can be any suitable adjuvant now known or later discovered.

In certain embodiments, the first container 110 can be temperature controlled. The first container 110 can be configured for storage of the active ingredient 112 at a temperature at a predetermined temperature for at least 24 hours. The predetermined temperature can be between about 2° C. and about 8° C. The first container 110 can be configured for storage of the active ingredient 112 at the predetermined temperature for at least 18 hours or for at least 12 hours.

The kit 100 can also include a third container 130 for temperature control, the third 130 container being insulated and configured to hold both the first container 110 and the second container 120 together with a cooling medium. The cooling medium can be at least one of water ice, dry ice, and a pre-refrigerated cooling gel pack. The kit 100 can include a syringe with an injection needle for the delivery of the compounded form upon being prepared. The kit 100 can include a fourth container for admixing the active ingredient 112 and the adjuvant to prepare the compounded form.

The first container 110 and the second container 120 can be vials. The first container 110 and the second container 120 can be connected with a breakable seal disposed between the active ingredient 112 and the adjuvant 122. A breaking of the breakable seal can permit the active ingredient 112 and the adjuvant to be admixed to prepare the compounded form. The first container 110 and the second container 120 can be provided in a form of a dual-chambered syringe.

Amounts of the active ingredient 112 in the first container 110 and the adjuvant 122 in the second container 120 can be configured to provide the active ingredient 112 in an amount between about 5% and about 50% of a total of the compounded form upon an entirety of the active ingredient 112 in the first container 110 being mixed with the entirety of the adjuvant 122 in the second container 120 to form the compounded form. Amounts of the active ingredient 112 in the first container 110 and the adjuvant 122 in the second container 120 can be configured to provide the active ingredient 112 in the amount between about 10% and about 40% of the total of the compounded form upon the entirety of the active ingredient 112 in the first container 110 being mixed with the entirety of the adjuvant 122 in the second container 120 to form the compounded form. The amounts of the active ingredient 112 in the first container 110 and the adjuvant 122 in the second container 120 can also be configured to provide the active ingredient 112 in the amount of about 30% of the total of the compounded form upon the entirety of the active ingredient 112 in the first container 110 being mixed with the entirety of the adjuvant 122 in the second container 120 to form the compounded form.

The active ingredient can be an allergen. The allergen can be selected from a group consisting of pollen, animal dander, dust mite, food, venom, autoantibody, and autologous tissue, as non-limiting examples. The active ingredient can be a purified substance from an allergen. The allergen can be selected from a group consisting of a recombinant protein and a synthesized peptide.

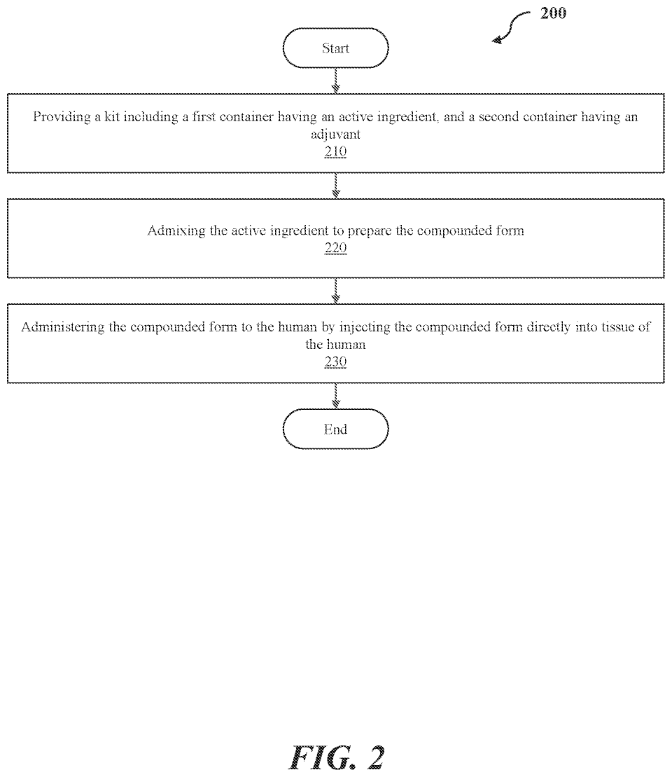

FIG. 2 is a flowchart that describes a method 200 for administering an active ingredient to a subject to elicit a response, according to some embodiments of the present disclosure. At a first step 210, the method can include providing a kit including a first container having an active ingredient with an active ingredient 112, and a second container having an adjuvant 122. At a second step 220, the method can include admixing the active ingredient 112 and the adjuvant 122 to prepare the compounded form. At a third step 230, the method can include administering the compounded form to the subject.

Where the active ingredient is an allergen, the compounded form can be administered by injecting the compounded form directly into the lymph node of the subject, injecting the compound subcutaneous, or other know or later discovered method of administering the compound form to the subject. The lymph node can be an axillary lymph node or an inguinal lymph node. In certain embodiments, the allergen can be delivered to an antigen presenting cell within the lymph node or an immune cell within the lymph node. The method can include a step of using an ultrasound device to monitor location of an injection needle. In further embodiments, the method can include a step of visualizing the lymph node using a radiological method.

Although the kit and method are described herein primarily with respect to immune modulating treatments involving allergies, it should be appreciated that the kit and the method can also be used for other types of immune modulating treatments, and that these other types are contemplated and considered to be within the scope of the present disclosure. Beyond modulating the immune system, for example, there are various physiological responses that one might aim to elicit through the administration of active ingredients. It may be desirable in certain embodiments to induce a hormonal response, such as the release of insulin to manage blood glucose levels in a diabetic patient. Another example is the stimulation of tissue repair and regeneration, which can be critical in wound healing or recovery from an injury. Additionally, the administration of certain compounds can be aimed at eliciting a neurotransmitter release to influence mood, cognitive function, or to alleviate a symptom of a neurological disorder. Pain relief is another significant physiological response, where the goal is to activate the body's endogenous analgesic systems to reduce the perception of pain. Furthermore, the induction of a metabolic response to influence the body's metabolism, such as increasing metabolic rate for weight management, is another example. Lastly, the promotion of vasodilation or vasoconstriction to regulate blood pressure and improve cardiovascular health is a response that can be targeted through specific active ingredients.

### EXAMPLES

Example embodiments of the present technology are provided with reference to the several figures enclosed herewith.

### Example 1

### Immune System Modulation

A kit 100 can be utilized to modulate the immune system's response to a specific allergen, such as pollen. The kit comprises two containers: the first container 110 includes an extract of the allergen, while the second container 120 contains an adjuvant, such as alum, which is known to enhance the immune response. The containers are designed to permit the mixing of the extract and the adjuvant to create a compounded form that is ready for administration. The first container 110 can be equipped with temperature control features, allowing the allergen extract is maintained at an optimal temperature, which can range between about 2° C. and about 8° C., to preserve its efficacy. This is particularly important for allergen extracts that can lose potency if not stored correctly.

When preparing for administration, a healthcare provider combines the allergen extract and the adjuvant in the specified proportions, creating a compounded form where the allergen makes up about 5% to 50% of the total volume. This formulation allows the allergen to be present in a concentration that is effective yet safe for the patient. The kit includes a syringe with an injection needle, or a dual-chambered syringe, which simplifies the process of mixing and allows for the direct injection of the compounded form into the patient's tissue. The administration of the compounded form can be performed by injecting it directly into a lymph node, such as an axillary or inguinal lymph node. The direct injection into the lymph node provides that the allergen extract is presented efficiently to the immune cells, such as antigen-presenting cells and lymphocytes, which are abundant in these nodes. This targeted delivery is intended to induce a desensitization effect, reducing the patient's hypersensitivity to the pollen allergen.

The treatment protocol can involve a series of injections, typically not exceeding three, administered over a period, such as once per month. Each injection contains a carefully measured dose of the allergen extract, starting with a low dose and potentially increasing in subsequent injections based on the patient's response. This gradual increase in dosage is a form of immunotherapy aimed at training the patient's immune system to become tolerant to the allergen, thereby reducing or eliminating the allergic response.

Throughout the treatment, the patient's immune response can be monitored. This can involve measuring specific IgG and IgE levels, changes in T-cell cytokine profiles, and conducting skin tests or controlled allergen exposures. The goal is to observe a shift in the immune response from an allergic reaction to a more regulated, non-reactive state. If successful, the patient will experience fewer or less severe allergic symptoms when naturally exposed to the allergen, such as during pollen season.

### Example 2

### Hormonal Modulation

A kit 100 can be adapted for use in modulating a hormonal response, specifically for the release of insulin to manage blood glucose levels in diabetic patients. The kit 100 includes two containers: the first container 110 contains an active ingredient, which in this case can be a compound that stimulates the body's natural insulin production or enhances insulin sensitivity. The second container 120 holds an adjuvant designed to augment the body's response to the active ingredient. The first container's temperature control feature can preserve the stability and bioactivity of the insulin-stimulating compound, which can be sensitive to temperature fluctuations. The first container 110 can maintain the compound at a predetermined temperature, typically between about 2° C. and about 8° C., for a minimum of 24 hours, such that the compound remains potent until the time of administration.

When preparing for administration, the healthcare professional can mix the insulin-stimulating compound with the adjuvant in the specified ratios to create a compounded form. This form is then ready for administration, with the active ingredient present in a concentration that is effective yet safe for the patient. The administration process involves injecting the compounded form directly into a tissue site that is conducive to the absorption and action of the insulin-stimulating compound, such as subcutaneous tissue known for its accessibility to insulin therapies. This targeted delivery ensures that the compound is introduced into the body in a manner that maximizes its potential to prompt the desired hormonal response, namely the release of insulin.

Specific examples of tissue sites that are conducive to the absorption and action of insulin-stimulating compounds include subcutaneous tissue, which is the layer of tissue directly under the skin, which is commonly used for insulin injections due to its rich blood supply that allows for consistent absorption of medications; abdominal fat, which often preferred for subcutaneous injections because the fat layer here tends to be more consistent and can facilitate a more predictable rate of absorption; upper arm, where the fatty tissue over the triceps area can be used for subcutaneous injections and can provide a convenient site for self-administration; upper thigh area, which provides another accessible site for subcutaneous injections, particularly for patients who can have less subcutaneous fat in other areas; buttocks, where fatty tissue is also a viable site for subcutaneous injections, although it can be less accessible for self-injection. These sites are typically recommended for the administration of insulin and insulin-stimulating compounds due to their ability to absorb the drug effectively and facilitate its action within the body.

Example active ingredients can include Sulfonylureas, Meglitinides, Incretin Mimetics (GLP-1 Agonists) and Dipeptidyl Peptidase-4 (DPP-4) Inhibitors. Example adjuvants include Glucagon-like Peptide-1 (GLP-1) Agonists, Thiazolidinediones (TZDs), Metformin, Alpha-Glucosidase Inhibitors, Chromium, Berberine, and Magnesium.

The treatment protocol can include a series of injections, typically not more than three, administered over a defined period. Each injection contains a predetermined dose of the insulin-stimulating compound, with the initial dose being conservative and potentially increasing in subsequent injections based on the patient's response and blood glucose monitoring. This approach aims to enhance the patient's endogenous insulin release or response, thereby improving glycemic control and reducing the reliance on exogenous insulin injections.

Throughout the treatment, the patient's hormonal response is closely monitored through regular blood glucose testing and possibly continuous glucose monitoring systems. The objective is to observe an improvement in blood glucose levels, indicating that the body's insulin response has been effectively modulated. If successful, the patient will experience better glycemic control, which is crucial in managing diabetes and militating against its associated complications.

### Example 3

### Tissue Repair and Regeneration for Wound Healing or Recovery from Injury

The kit 100 can be adapted for use in tissue repair and regeneration, particularly for wound healing or recovery from injury. The kit includes: the first container 110, which contains an active ingredient such as a growth factor or a peptide known to facilitate tissue repair, and the second container, which holds an adjuvant that enhances the healing response, potentially by modulating the local immune environment to support regeneration. The first container's temperature control is essential to maintain the integrity of the active ingredient, which can be a protein or peptide that is temperature-sensitive. By keeping the ingredient at a stable temperature, typically between about 2° C. and about 8° C., the kit allows the bioactive compound to retain its regenerative properties until it is ready for use.

Specific examples of growth factors or peptides that can be used as the active ingredient for tissue regeneration include: Platelet-Derived Growth Factor (PDGF), which is often used in wound healing, PDGF plays a role in attracting cells to the site of injury and stimulating their proliferation; Epidermal Growth Factor (EGF), which promotes epithelial cell growth, which can aid in the healing of skin and other epithelial tissues; Fibroblast Growth Factor (FGF), which can stimulate angiogenesis (formation of new blood vessels) and the proliferation of fibroblasts, which are essential for wound healing; Transforming Growth Factor-Beta (TGF-β), which is involved in the regulation of inflammation and stimulates the deposition of extracellular matrix, which is crucial for the strength and integrity of repaired tissue; Vascular Endothelial Growth Factor (VEGF), which is key in promoting the formation of new blood vessels, which is vital for supplying nutrients to healing tissues; Insulin-like Growth Factor (IGF), which can promote cell growth and differentiation, which aids in muscle and skeletal repair; Keratinocyte Growth Factor (KGF), which is used to stimulate the growth of keratinocytes, which are predominant in the epidermis of the skin; Bone Morphogenetic Proteins (BMPs), which are involved in bone formation and healing, making them particularly useful in orthopedic applications; Collagen Peptides, which can stimulate collagen synthesis, improving the strength and elasticity of the newly formed tissue; Matrix Metalloproteinase (MMP) Inhibitors, which can modulate the activity of enzymes that degrade the extracellular matrix, thus supporting tissue structure during healing. These growth factors and peptides can be used individually or in combination, depending on the specific requirements of the tissue repair process and the desired outcome of the treatment.

Example adjuvants can include growth factors, cytokines, extracellular matrix components, biological scaffold materials, immunomodulatory agents, toll-like receptor (TLR) agonists, microRNA, biomimetic peptides, platelet-rich plasma (PRP), mesenchymal stem cells (MSCs).

When it is time to administer the treatment, the healthcare provider can mix the active ingredient with the adjuvant in proportions to create a compounded form optimized for tissue regeneration. The administration involves injecting the compounded form directly into or around the damaged tissue, which can be a site of skin injury, a surgical wound, or an area of internal tissue damage. The direct delivery to the injury site can allow the active ingredient to be localized where it can exert its effects most effectively, promoting cell proliferation, angiogenesis, and matrix remodeling—all of which are crucial for tissue repair and regeneration.

The treatment protocol can involve a single injection or a series of injections, depending on the severity of the wound or injury and the patient's response to the initial treatment. Each injection can be dosed to provide an adequate amount of the active ingredient to stimulate healing without overstimulating the tissue, which can lead to fibrosis or scarring.

Throughout the treatment, the progress of wound healing or tissue regeneration is monitored through clinical assessments, imaging techniques, or biopsy samples, as appropriate. The aim is to observe signs of effective tissue repair, such as the reduction of wound size, the formation of new tissue, and the restoration of normal tissue architecture. If successful, the patient will experience accelerated healing, reduced recovery time, and improved functional outcomes.

### Example 4

### To Influence Mood, Cognitive Function, or to Alleviate Symptoms of Neurological Disorders

The kit 100 can be adapted for use in the field of neurology, specifically to influence mood, cognitive function, or to alleviate symptoms of neurological disorders such as depression, Alzheimer's disease, or Parkinson's disease. The kit includes two containers: the first container 110 holds an active ingredient, which can be a neurotrophic factor or a neuropeptide known to have neuroregenerative or neuromodulatory effects, and the second container 120 contains an adjuvant that enhances the therapeutic response of the nervous system to the active ingredient. The first container's temperature control allows for preserving the bioactivity of the neurotrophic factor or neuropeptide, which can be sensitive to temperature changes. By maintaining the compound at a stable temperature, typically between about 2° C. and about 8° C., the kit allowed that the neuroactive compound remains potent until administration.

Specific examples of neurotrophic factors or neuropeptides that can be used as the active ingredient for influencing mood, cognitive function, or alleviating symptoms of neurological disorders include: Brain-Derived Neurotrophic Factor (BDNF), which supports the survival of existing neurons and encourages the growth and differentiation of new neurons and synapses, particularly in the hippocampus, cortex, and basal forebrain—areas vital to learning, memory, and higher thinking; Nerve Growth Factor (NGF), which is critical for the survival and maintenance of sympathetic and sensory neurons and has been studied for its potential in treating neurodegenerative diseases like Alzheimer's; Glial cell line-Derived Neurotrophic Factor (GDNF), which promotes the survival of many types of neurons and is being researched for its therapeutic potential in Parkinson's disease and other neurodegenerative conditions; Ciliary Neurotrophic Factor (CNTF), which has been shown to reduce neuronal degeneration after injury and is being explored for its potential to treat diseases like multiple sclerosis and amyotrophic lateral sclerosis (ALS); Neuropeptide Y (NPY), which is involved in various brain functions, including anxiety, stress response, and feeding behavior, making it a potential target for mood disorders and obesity; Vasoactive Intestinal Peptide (VIP), which has a broad range of brain functions, including circadian rhythm regulation, and is being studied for its neuroprotective effects; Substance P, which is involved in the transmission of pain signals and is also implicated in mood and anxiety, making it a potential target for pain management and psychiatric disorders; Insulin-like Growth Factor 1 (IGF-1), which has neuroprotective and regenerative properties and is being investigated for its potential to improve cognitive deficits in conditions like brain injury and dementia; Oxytocin, which has the potential to treat various psychiatric disorders, including anxiety, depression, and autism spectrum disorders; Ghrelin, which neuroprotective effects and is being studied for its potential role in treating mood disorders and eating disorders. These neurotrophic factors and neuropeptides can be used individually or in combination, depending on the specific neurological condition being targeted and the desired therapeutic outcome.

Examples of the acceptable adjuvants include neurotrophic factors, anti-inflammatory agents, antioxidants, GABA agonists, cannabinoids, immunomodulators, ampakines, stem cell therapies, monoclonal antibodies, and metal chelators.

The administration process involves delivering the compounded form to a site where it can effectively reach the central nervous system and exert its effects on mood, cognitive function, or neurological symptoms. For example, intranasal delivery allows the neuroactive compound to bypass the blood-brain barrier and directly affect brain regions involved in mood regulation and cognitive processes. This targeted delivery ensures that the compound is introduced into the body in a manner that maximizes its potential to modulate neurological pathways.

The treatment protocol can involve a single administration or a series of administrations, depending on the severity of the neurological condition and the patient's response to the initial treatment. Each administration is carefully dosed to provide an adequate amount of the neuroactive compound to stimulate the desired therapeutic effect without causing adverse side effects. The goal is to achieve a balanced modulation of neural activity that improves mood, enhances cognitive function, or alleviates symptoms of neurological disorders.

Throughout the treatment, the patient's neurological response is closely monitored through clinical assessments, psychological evaluations, neuroimaging techniques, or biomarker analyses, as appropriate. The aim is to observe signs of improved neurological function, such as enhanced mood, better cognitive performance, or reduced symptoms of the targeted disorder. If successful, the patient will experience improved quality of life and potentially a reduction in the progression of the neurological condition.

### Example 5

### Pain Relief

The kit 100 can be adapted for application in pain management, specifically to activate the body's endogenous analgesic systems for pain relief. The kit 100 includes two primary components: the first container 110, which contains an active ingredient known to have analgesic properties, such as an opioid peptide or a non-opioid pain modulator, and the second container 120, which holds an adjuvant that enhances the pain-relieving response, potentially by modulating the local immune environment to support analgesia.

Examples of opioid peptides and non-opioid pain modulators that can be used as the active ingredient for pain relief include: Endorphins, which are endogenous opioid peptides produced by the central nervous system and pituitary gland, known for their potent analgesic effects and ability to produce a feeling of well-being; Enkephalins, which bind to opioid receptors and play a significant role in regulating nociception and pain relief; Dynorphins, which are involved in modulating pain and are thought to have a role in the body's natural response to stress and injury; Endomorphins, which have a high affinity for mu-opioid receptors and are known for their analgesic effects; Capsaicin, which is a non-opioid pain modulator that works by depleting substance P, a neuropeptide that transmits pain, and by desensitizing sensory neurons to pain; Cannabinoids, which can influence the endocannabinoid system to provide analgesic effects without the psychoactive effects of THC; Palmitoylethanolamide (PEA), which has been shown to have analgesic properties by reducing inflammation and pain through its action on the endocannabinoid system; Botulinum toxin, which can be used to reduce muscle spasticity and associated pain by inhibiting the release of acetylcholine at the neuromuscular junction; Clonidine, which can be used as an analgesic for certain types of pain due to its agonist action at alpha-2 adrenergic receptors, which inhibits the release of norepinephrine and dampens pain signaling; Ziconotide, which works by blocking calcium channels on nerve cells, thereby preventing the release of neurotransmitters that signal pain. These compounds can be used individually or in combination, depending on the specific pain condition being targeted and the desired therapeutic outcome.

Examples of acceptable adjuvants can include antidepressants, anticonvulsants, muscle relaxants, topical anesthetics, corticosteroids, Alpha-2 adrenergic agonists, NMDA receptor antagonists, calcium channel blockers, bisphosphonates, and cannabinoids.

When it is time to administer the treatment, the healthcare provider can mix the analgesic compound with the adjuvant in precise proportions to create a compounded form optimized for pain relief. The administration involves injecting the compounded form directly into or around the affected tissue, which can be a site of chronic pain such as arthritic joints, the back, or areas affected by neuropathic pain. The direct delivery to the pain site ensures that the analgesic compound is localized where it can exert its effects most effectively, promoting pain relief by activating the body's own pain control mechanisms, such as the release of endorphins or the inhibition of pain signal transmission.

The treatment protocol can involve a single administration or a series of administrations, depending on the severity of the pain and the patient's response to the initial treatment. Each administration is carefully dosed to provide an adequate amount of the analgesic compound to reduce pain perception without causing adverse side effects. The goal is to achieve a balanced analgesic response that provides effective pain relief and improves the patient's quality of life.

Throughout the treatment, the patient's pain levels, and overall response are closely monitored through pain scales, functional assessments, and potentially imaging techniques if applicable. The aim is to observe signs of effective pain relief, such as a decrease in reported pain levels, an increase in mobility, or a reduction in the use of additional analgesics. If successful, the patient will experience significant pain reduction, allowing for greater comfort and potentially a decrease in the reliance on systemic pain medications.

### Example 6

### Metabolic Rate Modulation

The kit 100 can be adapted for application in metabolic health, specifically to influence the body's metabolism in the context of weight management. The kit includes two containers: the first container 110 holds an active ingredient known to enhance metabolic rate, such as a peptide that stimulates thermogenesis or fat oxidation, and the second container 120 contains an adjuvant that boosts the metabolic response, potentially by enhancing the bioavailability or stability of the active ingredient.

Examples of active ingredients known to enhance metabolic rate, particularly peptides that stimulate thermogenesis or fat oxidation, include: Leptin, which is involved in regulating energy balance by inhibiting hunger, which in turn induces weight loss and increases energy expenditure; Glucagon-Like Peptide-1 (GLP-1), which is is an incretin hormone that stimulates insulin secretion but also has been shown to enhance lipolysis and thermogenesis; Fibroblast Growth Factor 21 (FGF21), which has been shown to increase energy expenditure and promote weight loss by enhancing thermogenesis and fatty acid oxidation; Growth Hormone-Releasing Hormone (GHRH), which can indirectly influence metabolism by stimulating the release of growth hormone, which has lipolytic effects; Peptide YY (PYY), which is released by the gut after eating and has been shown to decrease appetite and inhibit food intake, potentially increasing energy expenditure; Irisin, which is released in response to exercise and has been shown to induce the browning of white adipose tissue, leading to increased thermogenesis and improved metabolic rate; Melanotan II, which has been found to have appetite-suppressing effects, which can lead to weight loss and increased metabolic rate; Adiponectin, which is released from adipose tissue and is known to increase insulin sensitivity and fatty acid oxidation, leading to improved metabolic health; Thyrotropin-Releasing Hormone (TRH), which can stimulate metabolism by inducing the release of thyroid hormones, which are known to increase overall metabolic rate; Alpha-Melanocyte-Stimulating Hormone (α-MSH), which influences energy homeostasis and is involved in reducing food intake and increasing energy expenditure. These peptides can be used individually or in combination, depending on the specific metabolic health goals and the desired therapeutic outcome.

Example adjuvants can include Metformin, Alpha-Lipoic Acid, Carnitine, Chromium Picolinate, Coenzyme Q10 (CoQ10), Green Tea Extract, Fiber Supplements, Omega-3 Fatty Acids, Probiotics, and B Vitamins.

When preparing for administration, the healthcare professional mix the metabolic enhancer with the adjuvant in precise proportions to create a compounded form optimized for increasing the metabolic rate. The administration process involves delivering the compounded form to a site where it can effectively reach metabolic pathways and exert its effects on increasing the metabolic rate. For example, injection into abdominal adipose tissue allows the metabolic enhancer to interact directly with fat cells, promoting lipolysis and increasing energy expenditure. This targeted delivery ensures that the compound is introduced into the body in a manner that maximizes its potential to modulate metabolic processes, aiding in weight management.

The treatment protocol can involve a single administration or a series of administrations, depending on the patient's metabolic health goals and the response to the initial treatment. Each administration is carefully dosed to provide an adequate amount of the metabolic enhancer to stimulate an increase in metabolic rate without causing adverse side effects. The goal is to achieve a balanced metabolic response that supports weight management and potentially leads to weight loss.

Throughout the treatment, the patient's metabolic rate and overall response are closely monitored through clinical assessments, body composition analyses, and metabolic rate measurements, such as indirect calorimetry. The aim is to observe signs of an increased metabolic rate, such as a reduction in body fat percentage, an increase in lean muscle mass, or an improvement in markers of metabolic health. If successful, the patient will experience an enhancement in metabolic function, which can contribute to effective weight management and overall health improvement.

### Example 7

### Vasodilation and Blood Flow Modulation

The kit 100 can be adapted for application in cardiovascular health, specifically to regulate blood pressure through the promotion of vasodilation or vasoconstriction. The kit comprises two main components: the first container 110, which contains an active ingredient known to influence vascular tone, such as a peptide that induces vasodilation or vasoconstriction, and the second container 120, which holds an adjuvant that enhances the vascular response, potentially by stabilizing the active ingredient or modulating its interaction with vascular receptors.

Other examples of peptides that can be used to influence vascular tone and blood pressure include: Angiotensin II, which is a potent vasoconstrictor that plays a key role in the renin-angiotensin system, which regulates blood pressure and fluid balance; Bradykinin, which is a peptide that causes blood vessels to dilate (vasodilation), leading to a decrease in blood pressure; Atrial Natriuretic Peptide (ANP), which is released by the heart and induces vasodilation and diuresis, which can help to lower blood pressure; Vasopressin (Antidiuretic Hormone, ADH), which can cause vasoconstriction, which increases blood pressure, but it also has functions in retaining water in the kidneys, affecting blood volume and pressure; Endothelin-1, which is one of the most potent vasoconstrictors known and plays a significant role in the regulation of vascular tone; Calcitonin Gene-Related Peptide (CGRP), which is a potent vasodilator that can lower blood pressure by relaxing vascular smooth muscle; Adrenomedullin, which has strong vasodilatory effects, leading to decreased blood pressure and increased blood flow; Urotensin II, which is known for its role in vasoconstriction and can influence blood pressure regulation; Neuropeptide Y (NPY), which can act as a vasoconstrictor, particularly under sympathetic nervous system activation, contributing to increased blood pressure; Substance P, which is involved in the regulation of blood pressure through its vasodilatory actions, particularly in the context of inflammation. These peptides can be used individually or in combination, depending on the specific vascular health goals and the desired therapeutic outcome.

Examples of acceptable adjuvants can include Vasodilators, Angiogenesis Promoters, Antiplatelet Agents, Statins, Anticoagulants, Omega-3 Fatty Acids, Phosphodiesterase Inhibitors, Pentoxifylline, Flavonoids, and Ginkgo Biloba.

When it is time to administer the treatment, the healthcare provider mix the vasomodulator with the adjuvant in precise proportions to create a compounded form optimized for influencing blood pressure. The administration involves injecting the compounded form directly into or near the vascular tissue, which can be areas affected by hypertension or other cardiovascular conditions. For example, injection near the brachial artery allows the vasomodulator to act directly on the smooth muscle cells of the vessel wall, promoting vasodilation to lower blood pressure or vasoconstriction to raise blood pressure, as needed. This targeted delivery ensures that the compound is introduced into the body in a manner that maximizes its potential to modulate vascular tone, aiding in blood pressure regulation.

The treatment protocol can involve a single administration or a series of administrations, depending on the patient's cardiovascular health goals and the response to the initial treatment. Each administration is carefully dosed to provide an adequate amount of the vasomodulator to achieve the desired effect on blood pressure without causing adverse side effects. The goal is to achieve a balanced vascular response that supports cardiovascular health and potentially leads to the normalization of blood pressure levels.

Throughout the treatment, the patient's blood pressure and overall cardiovascular response are closely monitored through clinical assessments, blood pressure measurements, and potentially imaging techniques if applicable. The aim is to observe signs of improved blood pressure regulation, such as a reduction in hypertension, an improvement in blood flow, or a decrease in the risk of cardiovascular events. If successful, the patient will experience significant improvements in cardiovascular function, which can contribute to better health outcomes and potentially reduce the risk of heart disease and stroke.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments can be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

What is claimed is:

1. A kit for administering an active ingredient to a subject to elicit a response, comprising:
a first container having the active ingredient; and
a second container having an adjuvant;
wherein the first container and the second container are configured to permit for the active ingredient and the adjuvant to be admixed to prepare a compounded form for delivery of the active ingredient directly into tissue of the subject in a manner sufficient to elicit the response.

2. The kit of claim 1, wherein the active ingredient includes an allergen selected from a group consisting of pollen, animal dander, venom, dust mite, food, autoantibody, and autologous tissue.

3. The kit of claim 1, wherein the first container is temperature controlled.

4. The kit of claim 3, wherein the first container is configured for storage of the active ingredient at a predetermined temperature for at least 24 hours.

5. The kit of claim 4, wherein the predetermined temperature is between about 2° C. and about 8° C.

6. The kit of claim 4, wherein the first container is configured for storage of the active ingredient at the predetermined temperature for at least 18 hours.

7. The kit of claim 4, wherein the first container is configured for storage of the active ingredient at the predetermined temperature for at least 12 hours.

8. The kit of claim 3, further comprising a third container configured for temperature control, the third container being insulated, including a cooling medium, and configured to hold both the first container and the second container.

9. The kit of claim 8, wherein the cooling medium is at least one of water ice, dry ice, and a pre-refrigerated cooling gel pack.

10. The kit of claim 1, wherein amounts of the active ingredient in the first container and the adjuvant in the second container are configured to provide the adjuvant in an amount between about 5% and about 50% of a total of the compounded form upon an entirety of the active ingredient in the first container being mixed with an entirety of the adjuvant in the second container to form the compounded form.

11. The kit of claim 10, wherein amounts of the active ingredient in the first container and the adjuvant in the second container are configured to provide the adjuvant in the amount between about 10% and about 40% of the total of the compounded form upon the entirety of the active ingredient in the first container being mixed with an entirety of the adjuvant in the second container to form the compounded form.

12. The kit of claim 10, wherein the amounts of the active ingredient in the first container and the adjuvant in the second container are configured to provide the adjuvant in the amount of about 30% of the total of the compounded form upon the entirety of the active ingredient in the first container being mixed with an entirety of the adjuvant in the second container to form the compounded form.

13. The kit of claim 1, further comprising a syringe with an injection needle for the delivery of the compounded form upon being prepared.

14. The kit of claim 1, further comprising a fourth container for admixing the active ingredient and the adjuvant to prepare the compounded form.

15. The kit of claim 1, wherein the first container comprises a first vial and the second container comprises a second vial.

16. The kit of claim 1, wherein the first container and the second container are connected with a breakable seal disposed between the active ingredient and the adjuvant, whereby a breaking of the breakable seal permits the active ingredient and the adjuvant to be admixed to prepare the compounded form.

17. The kit of claim 1, wherein the first container comprises a first chamber and the second container comprises a second chamber of a dual-chambered syringe.

18. The kit of claim 1, wherein the physiological response is one of immune system modulation.

19. The kit of claim 18, wherein the adjuvant is alum.

20. A method for administering an active ingredient to a subject to elicit a response, the method comprising steps of:
providing a kit including a first container having an active ingredient, and a second container having an adjuvant, wherein the first container and the second container permit for the active ingredient and the adjuvant to be admixed to prepare a compounded form for delivery of the active ingredient directly into the subject in a manner sufficient to elicit the response;
admixing the active ingredient and the adjuvant to prepare the compounded form; and
administering the compounded form to the subject by injecting the compounded form directly into tissue of the subject.
